(19) United States
(12) Patent Application Publication
(10) Pub. No.: US 2021/0326526 A1
(43) Pub. Date: Oct. 21, 2021

(54) METHOD AND APPARATUS FOR GENOME SPELLING CORRECTION AND ACRONYM STANDARDIZATION

(71) Applicant: KONINKLIJKE PHILIPS N.V., EINDHOVEN (NL)

(72) Inventors: Charles Yee, Boston, MA (US); Samuel Frank Pilato, Cambridge, MA (US); Joseph Qin, Eindhoven (NL); Yi Zhen, Eindhoven (NL)

(21) Appl. No.: 17/252,811

(22) PCT Filed: Jun. 20, 2019
(86) PCT No.: PCT/EP2019/066322

§ 371 Date: Dec. 16, 2020

Related U.S. Application Data
(60) Provisional application No. 62/688,437, filed on Jun. 22, 2018.

(51) Int. Cl. G06F 40/232; G06F 40/242; G16B 50/10

(52) U.S. Cl. CPC G06F 40/232; G16B 50/10; G06F 40/242

(57) ABSTRACT

Various embodiments relate to a method and non-transitory computer readable medium for genome spelling correction, the method including the steps of performing pre-processing on a sentence, storing a first adjacent word to an unknown word and a second adjacent word to the unknown word, generating a plurality of candidate words for the unknown word, forming a plurality of trigrams with the first adjacent word to the unknown word and the second adjacent word to the unknown word and each of the plurality of candidate words, searching a trigram table for each of the plurality of trigrams and outputting the candidate word from the trigram with a highest trigram count in the trigram table.

### TECHNICAL FIELD

This disclosure relates generally to a spelling correction system, and more specifically, but not exclusively, to correcting misspelling of genes or acronyms.

### BACKGROUND

Automated and personalized clinical trial matching engines have been developed to help clinicians match patients to existing clinical trials that may benefit the patient. These systems may take patient data and use a machine learning model or search engines to identify clinical trials applicable to the patient. Sometimes words or technical terms the in descriptions clinical trials are misspelled making matching clinical trials to patients more difficult.

### SUMMARY

A brief summary of various embodiments is presented below. Embodiments address a method and apparatus for genome spelling correction and acronym standardization.

A brief summary of various example embodiments is presented. Some simplifications and omissions may be made in the following summary, which is intended to highlight and introduce some aspects of the various example embodiments, but not to limit the scope of the invention.

Detailed descriptions of example embodiments adequate to allow those of ordinary skill in the art to make and use the inventive concepts will follow in later sections.

Various embodiments relate to a method for genome spelling correction, the method including the steps of performing pre-processing on a sentence, storing a first adjacent word to an unknown word and a second adjacent word to the unknown word, generating a plurality of candidate words for the unknown word, forming a plurality of trigrams with the first adjacent word to the unknown word and the second adjacent word to the unknown word and each of the plurality of candidate words, searching a trigram table for each of the plurality of trigrams and outputting the candidate word from the trigram with a highest trigram count in the trigram table.

In an embodiment of the present disclosure, the method for genome spelling correction, the method including the steps of forming a plurality of bigrams with the first adjacent word and the second adjacent word to the unknown word and each of the plurality of candidate words, searching a bigram table for each of the plurality of bigrams and outputting the candidate word from the bigram with a highest bigram count in the bigram table.

In an embodiment of the present disclosure, the method for genome spelling correction, the method including the steps of forming a plurality of unigrams with each of the plurality of candidate words, searching a unigram table for each of the plurality of unigrams and outputting the candidate word from the unigram with the highest unigram count in the unigram table.

In an embodiment of the present disclosure, the trigram is formed in the order of the first adjacent word to the unknown word, at least one of the plurality of candidate words and the second adjacent word to the unknown word.

In an embodiment of the present disclosure, the trigram is formed in the order of at least one of the plurality of candidate words, the first adjacent word to the unknown word and the second adjacent word to the unknown word.

In an embodiment of the present disclosure, the trigram is formed in the order of the first adjacent word to the unknown word, the second adjacent word to the unknown word and at least one of the plurality of candidate words.

In an embodiment of the present disclosure, the plurality of candidate words are generated within edit distances 1 and 2 and compared with a dictionary.

In an embodiment of the present disclosure, the trigram table, the bigram table and the unigram table are formed from a database of plurality of trigrams, bigrams and unigrams extracted from text related to genomic data and wherein the table includes a count of the number of times each trigram, bigram, and unigram appears in the text related to genomic data.

Various embodiments relate to a non-transitory computer readable medium configured for genome spelling correction, the device including a memory and a processor configured to perform pre-processing on a sentence, store a first adjacent word to an unknown word and a second adjacent word to the unknown word, generate a plurality of candidate words for the unknown word, form a trigram with the first adjacent word to the unknown word and the second adjacent word to the unknown word and at least one of the plurality of candidate words, search for the trigram in a trigram table and output the candidate word from the trigram table with a highest trigram count.

In an embodiment of the present disclosure, the non-transitory computer readable medium configured for genome spelling correction, the device including the processor further configured to form a bigram with the first adjacent word to the unknown word and at least one of the plurality of candidate words, search for the bigram in a bigram table and output the candidate word from the bigram table with a highest bigram count.

In an embodiment of the present disclosure, the non-transitory computer readable medium configured for genome spelling correction, the device comprising the processor further configured to form a unigram with at least one of the plurality of candidate words, search for the unigram in the unigram table and output the candidate word from the unigram table with the highest unigram count.

In an embodiment of the present disclosure, the trigram is formed in the order of the first adjacent word to the unknown word, at least one of the plurality of candidate words and the second adjacent word to the unknown word.

In an embodiment of the present disclosure, the bigram is formed in the order of at least one of the plurality of candidate words and the first adjacent word to the unknown word.

In an embodiment of the present disclosure, the bigram is formed in the order of the first adjacent word to the unknown word and at least one of the plurality of candidate words.

In an embodiment of the present disclosure, the plurality of candidate words are generated within edit distances 1 and 2 and compared with a dictionary.

In an embodiment of the present disclosure, the trigram table, the bigram table and the unigram table are formed from a database of plurality of trigrams, bigrams and unigrams.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate example embodiments of concepts found in the claims and explain various principles and advantages of those embodiments.

These and other more detailed and specific features are more fully disclosed in the following specification, reference being had to the accompanying drawings, in which:

FIG. 1 illustrates a block diagram of modules in a system for genome spelling correction and acronym standardization;

FIG. 2 illustrates a flow diagram of the method for genome spelling correction and acronym standardization; and

FIG. 3 illustrates a block diagram of a real-time data processing system of the current embodiment.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments. Descriptors such as “first,” “second,” “third,” etc., are not meant to limit the order of elements discussed, are used to distinguish one element from the next, and are generally interchangeable.

However, a challenge emerged from the heterogeneous clinical trial descriptions data set. The trial match engine was based on ElasticSearch technology, which uses an inverted (word) index as search criteria.

By using an inverted (word) index as search criteria and as the accuracy of search results relies heavily on correctly spelled words within the index, misspelled words created a hurdle.

The trial matching engine uses as a query, for example, gene acronyms and amino acid substitution (biomarkers) or alpha-numeric arrangements that do not resemble typical or known English words. There are few, if any, agreed upon naming conventions between clinical trial institutions on how to spell certain gene acronyms and because of this and poor copyediting from the trial description authors, many trials may relate to a particular gene, but fail to mention that gene by the correct spelling.

Instead, a number of variations are written and indexed. Therefore, ElasticSearch may fail to match those descriptions containing the variants when the query is different, therefore, providing incomplete results.

These incomplete results are further compounded because almost every gene listed in the Human Genomic Nomenclature Society (“HUGO”) database has at least one synonym. Therefore, when a synonym is mentioned in a trial and the search query has only its canonical form, the trial will be missed in the search, constituting a false negative and leading to incomplete results.

ElasticSearch may apply a function named fuzzy word matching between the query and the indexed words, however, two issues remain. First, ElasticSearch's fuzzy words are, by default, calculated based on a Levenshtein distance of 0 for strings of up to two characters, 1 for strings up to five characters, and 2 for strings over five characters. However, this does not take into account gene acronyms, which are almost always under five characters, many of which require candidates from 2 or more Levenshtein distances. For example, PI3K to PIK3CA, HER2 to HER2/neu, MAG3 to MAGEA3, etc.

Levenshtein distances are calculated based on an unknown word. All candidates within the distances are looked up in a dictionary of known words. ElasticSearch does not contain domain specific dictionaries for gene acronyms and does not allow for gene synonym conversions because it lacks the look-up table capability.

Various National Institute of Health (“NIH”) funded databases include over 200,000 publicly and privately supported clinical studies involving human participants conducted around the world. Clinical trial descriptions listed by NIH are submitted from thousands of different pharmaceutical companies, research labs, hospitals, universities, and other institutions.

Many descriptions are cancer related treatments that make references to biomarkers and gene acronyms. However, due to the lack of agreement in conventions, the spellings used to identify a single biomarker can often differ between the various institutions responsible for providing trial descriptions.

Compounding the issue further, numerous cases of misspelling and arbitrary spacing/hyphenation in biomarkers in trial descriptions are present in the submitted clinical studies and the discrepancy in spelling poses an obstacle to trial matching using search engines and results in false negatives.

To prevent the deficiencies of ElasticSearch, a method is described herein to correct gene spelling and standardize gene acronyms so that multiple variants of one gene converges to its canonical spelling which will significantly improve recall rates of search.

In order to remedy the deficiencies, the method will resolve trial match performance reduction due to heterogeneous trial descriptions by correcting spelling of gene acronyms and biomarkers found in any document (i.e., a trial description), convert gene synonyms into their canonical form, support multiple dictionaries for reference where all dictionaries are plug-and-play compatible, and be fully customizable and allow for fine-tuning of various parameters from Levenshtein distances to word length thresholds to be considered a candidate for spelling correction.

By using a multi-layered domain specific spelling correction software which implements a hybrid of rule-based and statistical approaches in modem Natural Language Processing, clinical trial descriptions are groomed to contain standardized biomarkers and gene acronyms that are more accurate in the clinical sense, while maximizing true positives from querying through Elasticsearch algorithm.

This software will correct misspelled gene acronyms and biomarkers, convert gene synonyms to their canonical form, correct multiple genes or English words that are conjoined due to missing spaces, or with “-”, “/”, “(”, “)” inserted in random positions.

The spelling correction workflow utilizes an array of dictionary look-ups, disease and gene ontology, Bayesian language/error model and context sensitive selection based on legacy documents within the genomics domain.

Resolving these issues solves the effect of converging variant spellings into the one that is meant by the authors of the clinical trials and as a result, a search query with the canonical spelling will get all of the trials that contain any of its variants.

In the current embodiment, the software may correct English words, correct gene acronyms, amino acid substitution, and other biomarker signatures, convert synonyms of genes to their respective canonical form, detect commonly misspelled gene patterns and convert them into their correct canonical spelling, break up long strings where multiple English words have had the spaces between them truncated, correct English words found in space-truncated strings within 1 space edit distance, break up long biomarkers where gene names or gene and amino acid substitution have been truncated together, allow for customized dictionaries to let special words through (i.e., skip the correction), recognize conjoined words (allowing the option to skip them), recognize possessives (allow the option to skip them), recognize measurement units (allow the option to skip them), and recognize URLs and emails (allow the option to skip them).

The system includes two modules, the binary look-up module, and if a word is not found in any of the look-up tables, a Bayesian estimation module is applied to determine the most likely correction for that word.

Each document is processed line by line, meaning that a line is read and after the entire line is corrected, it is written into the output file. Each individual word within a line is first passed into a number of binary lookup steps. The words are passed in a left-to-right order.

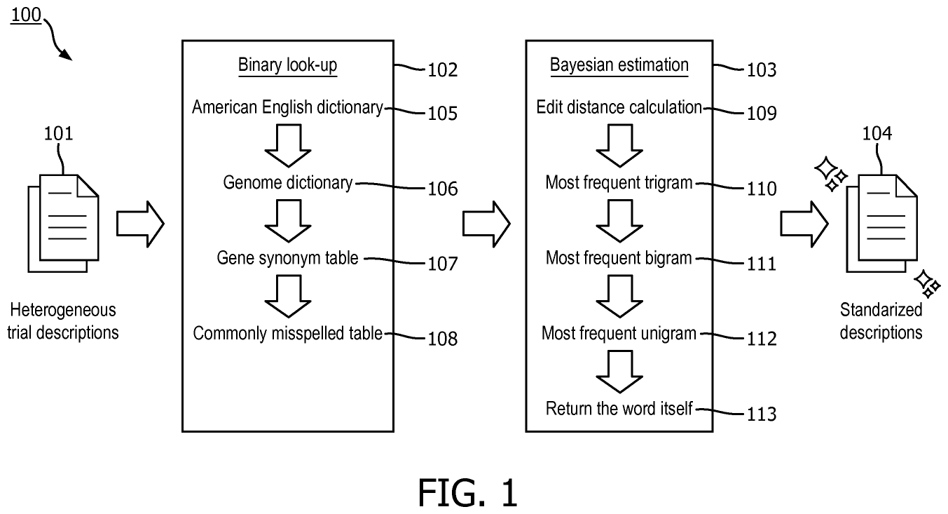

FIG. 1 illustrates a system 100 including heterogeneous trial descriptions 101 being input into a binary look-up module 102, then passed into a Bayesian estimation module 103 (if a word is not found in any of the binary look up tables), then output as a standardized description 104.

In short, when a word is found, no correction is made and the system 100 continues to the next word. When a word is not found in any of the dictionaries 105, 106 or tables 107, 108, it fails and continues onto the next stage, which is the Bayesian estimation 103.

A dictionary manager loads up all the dictionaries 105, 106, and the dictionary manager contains simple word checking functions such as checking to see if a word is in a particular dictionary, if a word is just the plural version of another word, if a world might be a possessive, if a word is a URL or an email address, if a word contains only punctuation or numeric, etc.

The word being passed into the binary look-up module 102 is first checked against a dictionary 105. For example, an Unbuntu American-English or any other dictionary. Both the original casing and an all lower-cased versions of the word are checked against a dictionary 105. If the word is found, then no correction is made and the system 100 moves onto the next word.

If the word is not found in the dictionary 102, the word is then checked against a list of canonical gene terms obtained from the HUGO Gene Nomanclature Committee, known as the genome dictionary 106. This check is case sensitive. If the word is found, then no correction is made, the word is written into the output file, and the system 100 moves onto the next word.

The system 100 maintains a conversion table of gene synonyms to a gene's canonical form. Whenever the word is found in the gene synonym table 107, the word is converted into its canonical form and written into the output file. This has the effect of unifying multiple synonym terms into one single canonical term and when the entirety of the clinical trials database is groomed using this output file, variant terms are replaced by a singular, canonical term, which has the effect of increasing the coverage of Elasticsearch when a canonical term is searched.

If the word is still not found, the system 100 proceeds to check the word against the commonly misspelled table 108. The commonly misspelled table 108 uses a different library sequence matcher to compare the differences in the number of characters divided by the total number of characters in the longer word.

Therefore, for every one of the 2,065 gene terms in clinical trial descriptions, other words found in trial descriptions are extracted that have the highest similarity scores to them. The frequency of each spelling variant, including the canonical terms, is also calculated.

The system 100, puts “1” in the max row and “0.9” in the min row (for similarity scores), and a list of canonical gene terms with the words similar to them are displayed. The UI tool allows the user to go inside of the individual trial descriptions and manually examine the occurrences of the potentially misspelled gene term. Once a user has determined that the similar term (potential misspelled) is a misspelling of the canonical term, that misspelling is added into commonly misspelled table 108.

For each entry in the commonly misspelled table 108, the misspelling is on the left of each line. Tab delimited on the right is the correct, canonical gene term. The system 100 will check within the commonly misspelled table 108 to determine if the word matches any of the misspellings in this commonly misspelled table 108, and if so, the correct canonical gene term is written into the output. The commonly misspelled table 108 allows flexibility in terms of the words to correct. As the contents of the commonly misspelled table 108 is data-driven based on occurrence frequency, with manual validation, it is reliable and can be incremented over time to be effective.

A word conversion module includes basic functionalities for building look-up tables, it is applicable to gene synonym table 107 and commonly misspelled table 108. The binary look-up module may also contain functions that looks up possible genes and amino acid substitutions where they may have been conjoined together.

If the word is not found in any of the dictionaries 105, 106 or tables 107, 108 in the binary look-up module, the system 100 proceeds to the Bayesian estimation module 103.

After the word passes through binary look-up module 102 and the word is still not found, the Bayesian estimation module 103 performs a method to “guess” what the correct spelling for that word is. A database of historically “correct” language is used.

The database used is the original dataset used by Norvig spelling correction, collected from the Penn Tree bank and Gutenberg project. Developed with the Penn Tree and Gutenberg data is 46M of sentences extracted from a large archive of medical journal on genomics. Each sentence in the database contains at least one gene.

The database is preprocessed by a generate Ngram module, where unigram, bigram, and trigrams are collected. generateNgram.py is a Python file which provides the functionalities to generate Ngrams given a text file.

There are a number of linguistic preprocessing which occur in the system 100 prior to the Ngram collection, and these preprocessing may be toggled on/off.

For example, another preprocessing feature is that ngrams are not collected across different sentences because any sentence may be followed by any other sentence, however, an individual word will likely be followed a narrower, more specific set of other words (e.g., “coca” and “cola”).

For example, another preprocessing feature is that lower casing is used to obtain a larger frequency count for a specific spelling.

For example, another preprocessing feature is that further splits from commas and semicolons and parenthesis are used for the same reason periods are skipped when collecting Ngrams.

For example, another preprocessing feature is that all other punctuation is removed.

For example, another preprocessing feature is that stop word removal is not active to conform with generateNgram.Norvig_train1 and generateNgram.Norvig_train2 collection conventions.

For example, another preprocessing feature is not using Porter Stemmer to stem each word. Stemming may affect some gene acronyms from being returned properly.

A porter stemmer module performs a standard stemmer allowing stemming feature when generating Ngrams.

For example, another preprocessing feature is not passing the words through the binary look-up module 102 first and before collecting Ngrams because processDescriptions.py pipeline handles dictionary check-ups. It is possible to only use the gene dictionary (instead of both English dictionary and gene dictionary) so that only domain specific words appear in the Ngram counts. However, this feature must be turned on when using bigrams and trigrams, as they need context with English words.

A process description module combines all the functionalities from other files together and takes words line by line, file by file, from a specified directory, uses binary look-up as well as Bayesian estimation to correct all words, then outputs the corrected version of documents into another directory with identical file names.

Once a sentence goes through preprocessing (not illustrated), the Ngrams from the sentence are collected. The Bayesian estimation 103 uses unigrams, bigrams, and trigrams.

Every time a word is not found in the dictionaries 105, 106 and the tables 107, 108 of the binary look-up module 102 and passed into Bayesian estimation module 103, the word before (previous_word) and the word after (next_word) to the unknown word are added. Additionally, trigrams may be formed using two previous words or the next two words with the unknown word.

In addition, within edit distances 1 and 2, all possible candidates of the unfound word that are spelled correctly (i.e., found in the dictionaries) are generated.

The combination of “previous_word candidate_word next_word” makes a trigram 110. This combination is searched from the trigram table 110 collected from the database. If it is found, the candidate word is returned that forms the trigram with the highest trigram count. Additionally, the trigram table may search for the other forms of trigrams as well. If no matching trigrams are found, the system 100 proceeds to searching the bigram table 111 collected from the database.

There are two types of possible bigrams: Forward bigrams and backward bigrams. A forward bigram is the combination of “previous_word candidate_word” and a backward bigram is “candidate_word next_word”.

For every candidate word for the unfound, the system 100 searches the database for the bigram (both forward and backward) that has the highest frequency count and returns the candidate word responsible for that bigram. If no matching bigrams are found, the system 100 proceeds to unigrams.

The system 100 searches for the candidate word, i.e., unigram, that has the highest count in the unigram table 112 and returns that candidate word as the correction.

The system 100 may detect possessives, measurement units, conjoined words, e-mail addresses and URL's, and exclude them from being spell corrected.

Because the gene synonym file contains over 80,000 gene synonyms, many of the synonyms span across multiple words. The system 100 uses a prefix tree to absorb all words needed to match a particular synonym in that list and return the canonical gene term.

The system 100 breaks up long strings where multiple English words have had the spaces between them deleted. In addition, some of the constituent words within the long string may have been misspelled.

The system 100 may recognize when two genes, or a gene and an amino acid substitution are malformed due to random punctuation in place of an expected space, or a missing space. (e.g., EGFR/ERBR, BRAFV600E). The system 100 may format the genes/amino acid substitutions into their constituent, well-formed parts (i.e., EGFR ERBR, BRAF V600E)

The system 100 may be implemented in software and may include various functions, including:

A generate Ngram module which provides the functionalities to generate Ngrams given a text file. There are seven preprocessing options for the Ngram generation outlined in the previous section Bayesian Estimation.

A dictionary manager loads all the dictionaries. The file contains simple word checking functions such as checking to see if a word is in a particular dictionary, if a word is just the plural version of another word, if a world might be a possessive, if a word is a URL or an email address, if a word contains only punctuation or numeric, etc.

genomeSpellCorrect.py which performs Bayesian estimation for an unknown word using Ngram tables.

PorterStemmer.py which uses standard stemmer allowing stemming feature when generating Ngrams.

processDescriptions.py which is a file which combines all the functionalities from other files together-takes words line by line, file by file, from a specified directory, uses binary look-up as well as Bayesian estimation to correct all words, then output the corrected version of documents into another directory with identical file names.

findSpellingErrorVariants.py which is a which provides utility functions to help generate possible misspellings when given a specific gene acronym/biomarker. The candidate misspellings are then looked up in clinical trial descriptions to see if their is a high frequency of a particular misspelling.

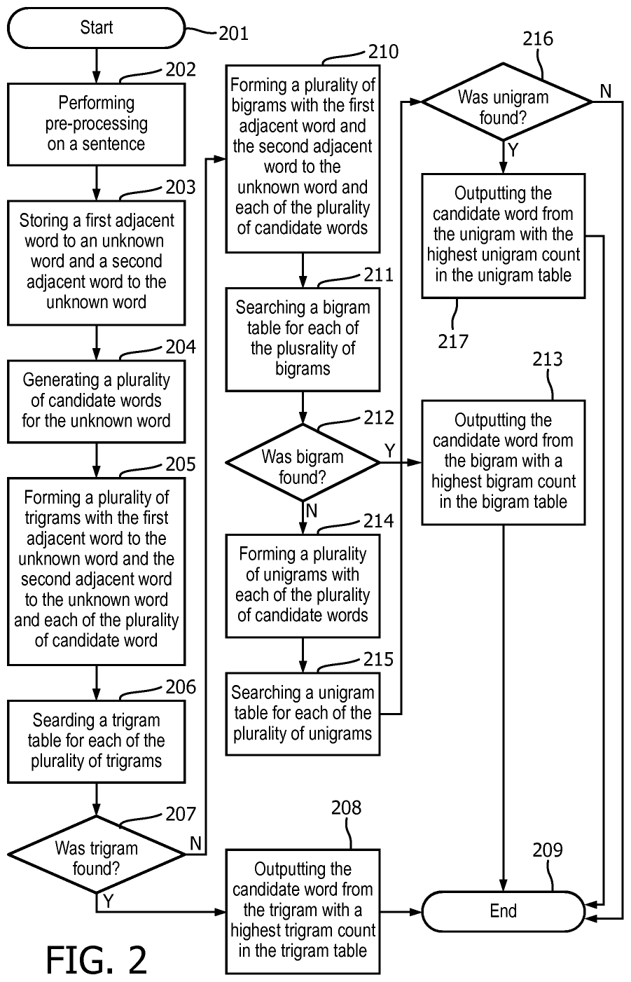

FIG. 2 illustrates a method 200 for genome spelling correction. The method begins at step 201.

The method 200 proceeds to step 202 which performs pre-processing on a sentence.

The method 200 then proceeds to step 203 which stores a first adjacent word to the unknown word and a second adjacent word to the unknown word.

The method 200 then proceeds to step 204 which generates a plurality of candidate words for the unknown word.

The method 200 then proceeds to step 205 which forms a plurality of trigrams with the first adjacent word, each one of the plurality of candidate words, and the second adjacent word. Note that trigrams may be formed with the candidate words in either the first, second, or third position of the trigram along with the appropriate adjacent words.

The method 200 then proceeds to step 206 which searches the trigram table for each of the plurality to trigrams.

The method 200 then proceeds to step 207 to determine whether any of the trigram were found. If yes, the method 200 proceeds to output the candidate word with the highest trigram count. The method 200 then proceeds to end at step 209.

If no, the method 200 proceeds to step 210 which forms a plurality of bigrams with the first adjacent word or the second adjacent word and each one of the plurality of candidate words.

The method 200 then proceeds to step 211 which searches for the bigram table for each of the bigrams.

The method 200 then proceeds to step 212 which determines whether any of the plurality of the bigrams were found in the bigram table. If yes, the method 200 proceeds to output the candidate word with the highest bigram count. The method 200 then proceeds to end at step 209.

If no, the method 200 proceeds to step 214 which forms a plurality of unigrams from the plurality of candidate words.

The method 200 then proceeds to step 215 which searches the unigram table for the plurality of unigrams.

The method 200 then proceeds to step 216 which determines whether any of the plurality of unigrams were found. If yes, the method 200 proceeds to step 217 which outputs the candidate word with the highest unigram count. The method 200 then proceeds to end at step 209.

If no, the method proceeds to end at step 209.

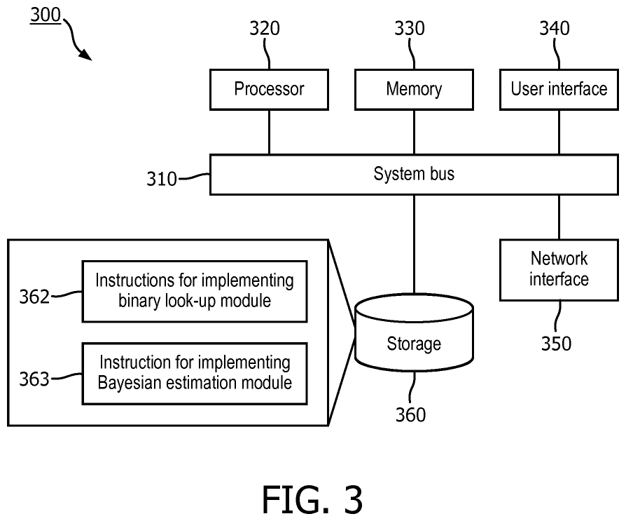

FIG. 3 illustrates an exemplary hardware diagram 300 for implementing a method for genome spelling correction, using a Bayesian estimation. As shown, the device 300 includes a processor 320, memory 330, user interface 340, network interface 350, and storage 360 interconnected via one or more system buses 310. It will be understood that FIG. 1 constitutes, in some respects, an abstraction and that the actual organization of the components of the device 300 may be more complex than illustrated.

The processor 320 may be any hardware device capable of executing instructions stored in memory 330 or storage 360 or otherwise processing data. As such, the processor may include a microprocessor, field programmable gate array (FPGA), application-specific integrated circuit (ASIC), or other similar devices.

The memory 330 may include various memories such as, for example L1, L2, or L3 cache or system memory. As such, the memory 330 may include static random access memory (SRAM), dynamic RAM (DRAM), flash memory, read only memory (ROM), or other similar memory devices.

The user interface 340 may include one or more devices for enabling communication with a user such as an administrator. For example, the user interface 340 may include a display, a mouse, and a keyboard for receiving user commands. In some embodiments, the user interface 340 may include a command line interface or graphical user interface that may be presented to a remote terminal via the network interface 350.

The network interface 350 may include one or more devices for enabling communication with other hardware devices. For example, the network interface 350 may include a network interface card (NIC) configured to communicate according to the Ethernet protocol. Additionally, the network interface 350 may implement a TCP/IP stack for communication according to the TCP/IP protocols. Various alternative or additional hardware or configurations for the network interface 350 will be apparent.

The storage 360 may include one or more machine-readable storage media such as read-only memory (ROM), random-access memory (RAM), magnetic disk storage media, optical storage media, flash-memory devices, or similar storage media. In various embodiments, the storage 360 may store instructions for execution by the processor 320 or data upon with the processor 320 may operate. For example, the storage 360 may store instructions for implementing the binary look-up module 362 and instructions for implementing the Bayesian estimation module 363.

It will be apparent that various information described as stored in the storage 360 may be additionally or alternatively stored in the memory 330. In this respect, the memory 330 may also be considered to constitute a “storage device” and the storage 360 may be considered a “memory.” Various other arrangements will be apparent. Further, the memory 330 and storage 360 may both be considered “non-transitory machine-readable media.” As used herein, the term “non-transitory”will be understood to exclude transitory signals but to include all forms of storage, including both volatile and non-volatile memories.

While the host device 300 is shown as including one of each described component, the various components may be duplicated in various embodiments. For example, the processor 320 may include multiple microprocessors that are configured to independently execute the methods described herein or are configured to perform steps or subroutines of the methods described herein such that the multiple processors cooperate to achieve the functionality described herein. Further, where the device 300 is implemented in a cloud computing system, the various hardware components may belong to separate physical systems. For example, the processor 320 may include a first processor in a first server and a second processor in a second server.

It should be apparent from the foregoing description that various exemplary embodiments of the invention may be implemented in hardware. Furthermore, various exemplary embodiments may be implemented as instructions stored on a non-transitory machine-readable storage medium, such as a volatile or non-volatile memory, which may be read and executed by at least one processor to perform the operations described in detail herein. A non-transitory machine-readable storage medium may include any mechanism for storing information in a form readable by a machine, such as a personal or laptop computer, a server, or other computing device. Thus, a non-transitory machine-readable storage medium may include read-only memory (ROM), random-access memory (RAM), magnetic disk storage media, optical storage media, flash-memory devices, and similar storage media and excludes transitory signals.

It should be appreciated by those skilled in the art that any blocks and block diagrams herein represent conceptual views of illustrative circuitry embodying the principles of the invention. Implementation of particular blocks can vary while they can be implemented in the hardware or software domain without limiting the scope of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Accordingly, it is to be understood that the above description is intended to be illustrative and not restrictive. Many embodiments and applications other than the examples provided would be apparent upon reading the above description. The scope should be determined, not with reference to the above description or Abstract below, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

1. A computer-implemented method for correction of a genomic term, the method comprising the steps of:
performing pre-processing on a sentence;
storing a first adjacent word to an unknown word and a second adjacent word to the unknown word;
generating a plurality of candidate words for the unknown word;
forming a plurality of trigrams with the first adjacent word to the unknown word and the second adjacent word to the unknown word and each of the plurality of candidate words;
searching a trigram table for each of the plurality of trigrams; and
outputting the candidate word from the trigram with a highest trigram count in the trigram table.

2. The computer-implemented method for correction of a genomic term of claim 1, the method comprising the steps of:
forming a plurality of bigrams with the first adjacent word and the second adjacent word to the unknown word and each of the plurality of candidate words;
searching a bigram table for each of the plurality of bigrams; and
outputting the candidate word from the bigram with a highest bigram count in the bigram table.

3. The computer-implemented method for correction of a genomic term of claim 2, the method comprising the steps of:
forming a plurality of unigrams with each of the plurality of candidate words;
searching a unigram table for each of the plurality of unigrams; and
outputting the candidate word from the unigram with the highest unigram count in the unigram table.

4. The computer-implemented method for correction of a genomic term of claim 1, wherein the plurality of trigrams are formed in the order of the first adjacent word to the unknown word, at least one of the plurality of candidate words and the second adjacent word to the unknown word.

5. The computer-implemented method for correction of a genomic term of claim 1, wherein the plurality of trigrams are formed in the order of at least one of the plurality of candidate words, the first adjacent word to the unknown word and the second adjacent word to the unknown word.

6. The computer-implemented method for correction of a genomic term of claim 1, wherein the plurality of trigrams are formed in the order of the first adjacent word to the unknown word, the second adjacent word to the unknown word and at least one of the plurality of candidate words.

7. The computer-implemented method for correction of a genomic term of claim 1, wherein the plurality of candidate words are generated within edit distances 1 and 2 and compared with a dictionary.

8. The computer-implemented method for correction of a genomic term of claim 3, wherein the trigram table, the bigram table and the unigram table are formed from a database of plurality of trigrams, bigrams and unigrams extracted from text related to genomic data and wherein the table includes a count of the number of times each trigram, bigram, and unigram appears in the text related to genomic data.

9. A non-transitory computer readable medium configured for correction of a genomic term, the device comprising:
a memory; and
a processor configured to:
perform pre-processing on a sentence;
store a first adjacent word to an unknown word and a second adjacent word to the unknown word;
generate a plurality of candidate words for the unknown word;
form a plurality of trigrams with the first adjacent word to the unknown word and the second adjacent word to the unknown word and at least one of the plurality of candidate words;
search for each of the plurality trigrams in a trigram table, and
output the candidate word from the trigram table with a highest trigram count.

10. The non-transitory computer readable medium configured for correction of a genomic term of claim 9, the device comprising:
the processor further configured to:
form a plurality of bigrams with the first adjacent word to the unknown word and at least one of the plurality of candidate words;
search for the bigram in a bigram table;
output the candidate word from the bigram table with a highest bigram count.

11. The non-transitory computer readable medium configured for correction of a genomic term of claim 10, the device comprising:
the processor further configured to:
form a plurality of unigram with at least one of the plurality of candidate words;
search for each of the unigram in the unigram table;
output the candidate word from the unigram table with the highest unigram count.

12. The non-transitory computer readable medium configured for correction of a genomic term of claim 9, wherein the plurality of trigrams are formed in the order of the first adjacent word to the unknown word, at least one of the plurality of candidate words and the second adjacent word to the unknown word.

13. The non-transitory computer readable medium configured for correction of a genomic term of claim 10, wherein the plurality of bigrams are formed in the order of at least one of the plurality of candidate words and the first adjacent word to the unknown word.

14. The non-transitory computer readable medium configured for correction of a genomic term of claim 10, wherein the plurality of bigrams are formed in the order of the first adjacent word to the unknown word and at least one of the plurality of candidate words.

15. (canceled)

16. The non-transitory computer readable medium configured for correction of a genomic term of claim 11, wherein the trigram table, the bigram table and the unigram table are formed from a database of plurality of trigrams, bigrams and unigrams.
